SEQUENCE LISTING (5 sequences)

SEQ ID NO: 1 - DNA, 2608 bp, Arabidopsis thaliana
gtcgctctctttgcccatctctcatctccactcttctcattccgtaagtaaaaaaacaaaaaacaaacaaaaatggcttcccacattgttggatatccacgtatgggacctaagagagagctcaagtttgcattggagtctttctgggatggcaagagcagtgccgatgatttgcagaaggtgtctgctgatctcaggtctgatatctggaaacagatgtctgctgctgggattaagtatatcccaagcaacaccttttctcattatgaccaggtgcttgacaccaccgccatgcttggtgctgttccatctagatatggatttaccagtggtgagatcggtctcgatgtttacttctccatggctagaggaaatgcctctgttccagctatggagatgaccaagtggtttgacaccaactaccattacatcgtcccagagttgggccctgaagtgaaattttcttacgcatctcacaaggctgtcaatgagtacaaggaggccaaggctcttggtgttgagaccgtccctgtacttgttggccctgtctcttacttgcttctttccaagcttgctaagggtgttgacaagtcatttgatcttctctcccttctccccaaaatcctcccagtttacaaggaagtcattgcagagcttaaggcagctggtgcctcctggattcagcttgatgagcctctctttgtcatggatctcgagggtcacaaactccaggcttttagcggtgcctatgctgagcttgaatcaactctctctggtctgaatgttcttgtggagacctacttcgctgatatccctgctgaagcatacaagacccttacttccttgaagggtgtgactgccttcggatttgatttggttcgtggcaccaagaccattgacttgatcaagtcaggtttcccacagggcaagtacctctttgctggtgttgttgacggaaggaacatctgggccaatgacctcgctgcctctctcatcaccttgcagtcacttgagggtgttgttggtaaagacaagcttgtggtctcaacctcttgctctcttctccacactgccgttgaccttattaacgagactaagcttgatgctgaaatcaagtcgtggctagcttttgctgcccagaaggttgttgaagttgacgcattggccaaggctttggccggtcagacaaatgagagtttcttcactgccaacgctgacgcattgtcttcgaggaggtcttccccaagagtcaccaatgagtctgtccagaaggctgctgctgctttgaagggatctgaccaccgccgtacaactgaagttagcgcaaggctagatgctcagcagaagaagcttaaccttccaatcctcccaaccacaaccattggatccttcccacagaccgtggaactcaggagagttcgccgtgaatacaaggccaagaaaatctctgaagaggattacgtcaaggccatcaaggaagagatcaagaaagttgttgacatccaagaggaccttgacattgatgttcttgttcacggagagcctgagagaaacgacatggttgagtactttggagagcaattgtcaggtttcgcattcacagcaaacggatgggtgcaatcctatggatctcgctgtgtgaagccaccagttatctatggtgacgtgagccgccccaagccaatgacagtcttctggtcctcaacagctcagagcatgaccaaacgtccaatgaagggtatgcttacaggtccagtcacaattctcaactggtcttttgtcagaaacgaccagcccaggcacgaaacctgttaccagatcgctttggccatcaaagatgaagtggaagacctcgagaaaggcggtattggagtcattcagatcgatgaagccgcacttagagaaggattgcctcttaggaaagccgaacactctttctacttggactgggctgttcactctttcagaatcaccaactgtggcgtccaagacagcactcagattcacactcacatgtgttactcaaacttcaacgacatcatccactcaatcattgacatggacgctgatgtcatcaccattgagaactctcgttcagacgagaagcttctctcagtgttccgtgaaggagtgaagtacggtgcaggaatcggtcctggtgtttacgacattcactctccgagaataccatccacagatgaaattgcagacaggatcaacaagatgcttgcggttcttgagcagaacatcttgtgggttaaccctgactgtggtctgaagacaaggaagtacactgaggttaaaccagcacttaaagccatggttgacgcggctaagcttatccgctcccagctcggtagtgccaagtgaagagcttgaagatattatttctatattccgggatttttctacgtggtttgtgtttgttcagtttcaataacttttcttccaagaaaaatattttagccaaagttaggttttgagggaatggagtcacactctctcgctttcgttgaagagagtttacggctttatactatatgtttctcttgttgcaatgttatatgtatctttgttttctctaatgaaatatatgcttctttgatct

SEQ ID NO: 2 - protein, 765 aa, Arabidopsis thaliana
MASHIVGYPRMGPKRELKFALESFWDGKSSADDLQKVSADLRSDIWKQMSAAGIKYIPSNTFSHYDQVLDTTAMLGAVPSRYGFTSGEIGLDVYFSMARGNASVPAMEMTKWFDTNYHYIVPELGPEVKFSYASHKAVNEYKEAKALGVETVPVLVGPVSYLLLSKLAKGVDKSFDLLSLLPKILPVYKEVIAELKAAGASWIQLDEPLFVMDLEGHKLQAFSGAYAELESTLSGLNVLVETYFADIPAEAYKTLTSLKGVTAFGFDLVRGTKTIDLIKSGFPQGKYLFAGVVDGRNIWANDLAASLITLQSLEGVVGKDKLVVSTSCSLLHTAVDLINETKLDAEIKSWLAFAAQKVVEVDALAKALAGQTNESFFTANADALSSRRSSPRVTNESVQKAAAALKGSDHRRTTEVSARLDAQQKKLNLPILPTTTIGSFPQTVELRRVRREYKAKKISEEDYVKAIKEEIKKVVDIQEDLDIDVLVHGEPERNDMVEYFGEQLSGFAFTANGWVQSYGSRCVKPPVIYGDVSRPKPMTVFWSSTAQSMTKRPMKGMLTGPVTILNWSFVRNDQPRHETCYQIALAIKDEVEDLEKGGIGVIQIDEAALREGLPLRKAEHSFYLDWAVHSFRITNCGVQDSTQIHTHMCYSNFNDIIHSIIDMDADVITIENSRSDEKLLSVFREGVKYGAGIGPGVYDIHSPRIPSTDEIADRINKMLAVLEQNILWVNPDCGLKTRKYTEVKPALKAMVDAAKLIRSQLGSAK

SEQ ID NO: 3 - DNA, 2298 bp, Arabidopsis thaliana
atggcttcccacattgttggatatccacgtatgggacctaagagagagctcaagtttgcattggagtctttctgggatggcaagagcagtgccgatgatttgcagaaggtgtctgctgatctcaggtctgatatctggaaacagatgtctgctgctgggattaagtatatcccaagcaacaccttttctcattatgaccaggtgcttgacaccaccgccatgcttggtgctgttccatctagatatggatttaccagtggtgagatcggtctcgatgtttacttctccatggctagaggaaatgcctctgttccagctatggagatgaccaagtggtttgacaccaactaccattacatcgtcccagagttgggccctgaagtgaaattttcttacgcatctcacaaggctgtcaatgagtacaaggaggccaaggctcttggtgttgagaccgtccctgtacttgttggccctgtctcttacttgcttctttccaagcttgctaagggtgttgacaagtcatttgatcttctctcccttctccccaaaatcctcccagtttacaaggaagtcattgcagagcttaaggcagctggtgcctcctggattcagcttgatgagcctctctttgtcatggatctcgagggtcacaaactccaggcttttagcggtgcctatgctgagcttgaatcaactctctctggtctgaatgttcttgtggagacctacttcgctgatatccctgctgaagcatacaagacccttacttccttgaagggtgtgactgccttcggatttgatttggttcgtggcaccaagaccattgacttgatcaagtcaggtttcccacagggcaagtacctctttgctggtgttgttgacggaaggaacatctgggccaatgacctcgctgcctctctcatcaccttgcagtcacttgagggtgttgttggtaaagacaagcttgtggtctcaacctcttgctctcttctccacactgccgttgaccttattaacgagactaagcttgatgctgaaatcaagtcgtggctagcttttgctgcccagaaggttgttgaagttgacgcattggccaaggctttggccggtcagacaaatgagagtttcttcactgccaacgctgacgcattgtcttcgaggaggtcttccccaagagtcaccaatgagtctgtccagaaggctgctgctgctttgaagggatctgaccaccgccgtacaactgaagttagcgcaaggctagatgctcagcagaagaagcttaaccttccaatcctcccaaccacaaccattggatccttcccacagaccgtggaactcaggagagttcgccgtgaatacaaggccaagaaaatctctgaagaggattacgtcaaggccatcaaggaagagatcaagaaagttgttgacatccaagaggaccttgacattgatgttcttgttcacggagagcctgagagaaacgacatggttgagtactttggagagcaattgtcaggtttcgcattcacagcaaacggatgggtgcaatcctatggatctcgctgtgtgaagccaccagttatctatggtgacgtgagccgccccaagccaatgacagtcttctggtcctcaacagctcagagcatgaccaaacgtccaatgaagggtatgcttacaggtccagtcacaattctcaactggtcttttgtcagaaacgaccagcccaggcacgaaacctgttaccagatcgctttggccatcaaagatgaagtggaagacctcgagaaaggcggtattggagtcattcagatcgatgaagccgcacttagagaaggattgcctcttaggaaagccgaacactctttctacttggactgggctgttcactctttcagaatcaccaactgtggcgtccaagacagcactcagattcacactcacatgtgttactcaaacttcaacgacatcatccactcaatcattgacatggacgctgatgtcatcaccattgagaactctcgttcagacgagaagcttctctcagtgttccgtgaaggagtgaagtacggtgcaggaatcggtcctggtgtttacgacattcactctccgagaataccatccacagatgaaattgcagacaggatcaacaagatgcttgcggttcttgagcagaacatcttgtgggttaaccctgactgtggtctgaagacaaggaagtacactgaggttaaaccagcacttaaagccatggttgacgcggctaagcttatccgctcccagctcggtagtgccaagtga

SEQ ID NO: 4 - DNA, 29 bp, Artificial Sequence
  Sense Primer
ggtaaccgtcgctctctttgcccatctct

SEQ ID NO: 5 - DNA, 31 bp, Artificial Sequence
  Anti-sense Primer
agatctagatcaaagaagcatatatttcatt

(19) United States
(12) Patent Application Publication
(10) Pub. No.: US 2010/0037345 A1
(43) Pub. Date: Feb. 11, 2010

(54) Polypeptide having Methionine Synthesis Function, Polynucleotide Encoding the Polypeptide, and Those Use

(75) Inventors: Dong Hee Lee, Busan (KR); Tae-hoon Kim, Busan (KR); Kook Jin Kim, Gyeongsangbuk-do (KR); Jong Bo Kim, Gyeongsangbuk-do (KR); Kyung Mok Park, Gyeongsangbuk-do (KR); In Taek Hwang, Daejeon (KR); No Joong Park, Daejeon (KR)

Correspondence Address: INTELLECTUAL PROPERTY LAW GROUP LLP, 12 SOUTH FIRST STREET, SUITE 1205, SAN JOSE, CA, 95113, US

(73) Assignees: GENOMINE, INC., Pohang-si, Gyeongsangbuk-do (KR); KOREA RESEARCH INSTITUTE OF CHEMICAL TECHNOLOGY, Daejeon (KR)

(21) Appl. No.: 12/520,528

(22) PCT Filed: Dec. 20, 2007
(86) PCT No.: PCT/KR2007/006673

§ 371 Date: Jun. 19, 2009

(30) Foreign Application Priority Data
Dec. 19, 2006 (KR) 10-2006-0129747

(51) Int. Cl. C12N 15/82; C12N 9/00; C12N 15/11; C12N 15/00; C12N 1/21; C12Q 1/00

(52) U.S. Cl. 800/278; 435/183; 536/23.2; 536/24.5; 435/320.1; 435/252.3; 435/4

(57) ABSTRACT

Disclosed herein are a polypeptide having a methionine synthesis function, a polynucleotide encoding the same, and uses thereof.

### TECHNICAL FIELD

The present invention relates to a polypeptide having a methionine synthesis function, a polypeptide coding for the polypeptide, and the use thereof.

### BACKGROUND ART

Methionine is an essential sulfur-containing amino acid for all living organisms, and its derivative S-adenosyl methionine (SAM) serves as an activated methyl donor.

Methionine biosynthesis can be divided into two stages.

A first phase is the conversion of cysteine into homocysteine, which is catalyzed by cystathionine synthase and cystathionine lyase.

In a second stage, the subsequent methylation of the thiol group of homocysteine, which is catalyzed by methionine synthase, affords methionine. In addition to being involved in methionine biosynthesis, those enzymes are known to be responsible for the recovery of the methyl group of S-adenosylmethionine after methylation (Ravanel S et al, PNAS 95: 7805-7812, 1998). Some of these methionine biosynthesis enzymes require vitamin B12 as a cofactor, whereas others do not. That is, there are vitamin B12 (cobalamin)-dependent and vitamin B12-independent enzymes. Interestingly, the vitamin B12-independent enzymes are not found in humans or animals, but exist only in plants (Eichel J et al, Eur J Biochem 230: 1053-1058, 1995).

An essential amino acid, methionine is not synthesized in humans and animals due to the lack of the vitamin B12-independent methionine synthesis enzymes. There is great significance in the fact that the vitamin B12-dependent methionine synthesis enzymes exist in plants, but not in humans or animals. This implies that given that any vitamin B12-independent enzyme plays a pivotal role in methionine biosynthesis, an inhibitor thereof may be used to regulate plant growth, e.g., to kill plants, without injuring humans or animals.

For this reason, botanists have made a great effort to find a vitamin B12-independent enzyme that is essentially responsible for methionine biosynthesis.

Under this background, the present invention has been accomplished.

### DISCLOSURE

### Technical Problem

It is therefore an object to provide a polypeptide serving as a vitamin B12-independent methionine synthesis enzyme indispensable for methionine biosynthesis.

It is another object of the present invention to provide a polynucleotide coding for the polypeptide.

It is a further object of the present invention to provide an antisense nucleotide sequence complementary to the polynucleotide.

It is still a further object of the present invention to provide a method for inhibiting plants from growing.

It is still another object of the present invention to provide a method of screening a plant growth inhibitor.

It is yet another object of the present invention to provide an inhibitor of plant growth, identified by the screening method.

### Technical Solution

In order to accomplish the above objects, experiments were conducted as described in the Example section, below. In brief, using the primers synthesized on the basis of a putative vitamin B12-independent methionine synthesis enzyme protein (GenBank accession number NM 180176) of Arabidopsis thaliana, a full-length cDNA including 5′- and 3′-UTR was obtained from Arabidopsis thaliana. A recombinant vector, in which the cDNA was cloned in an antisense direction, was transformed into Arabidopsis thaliana. This mutant Arabidopsis thaliana was observed to have distorted and discolored leaves, was significantly inhibited from growing, and finally died. Further, the mutant plant was found to be a methionine auxotroph that recovered its wild-type phenotype upon methionine treatment.

These data obtained from the experiments imply that the putative enzyme obtained by the present inventors is a vitamin B12-independent methionine indispensable for methionine biosynthesis.

Based on these experimental data, the present invention is provided.

In accordance with an aspect thereof, the present invention provides a polypeptide serving as a vitamin B12-independent methionine synthesis enzyme essential for methionine biosynthesis.

In greater detail, the polypeptide serving as a vitamin B12-independent methionine synthesis enzyme having an essential function for methionine biosynthesis is one of the following polypeptides.

(a) a polypeptide having the entire amino acid sequence of SEQ. ID. NO. 2;

(b) a polypeptide containing a substantial part of the amino acid sequence of SEQ. ID. NO. 2; and

(c) a polypeptide substantially similar to that of (a) or (b).

As used in the foregoing and the following descriptions including the claims, the phrase “having an essential function for methionine biosynthesis” is intended to mean that when the polypeptide of the present invention is not produced, or is inactivated, methionine biosynthesis is inhibited to the extent that plants are inhibited from growing. With regard to the inhibition of plant growth, this will be elucidated below.

As used in the foregoing and the following descriptions, including the claims, the phrase or term “a polypeptide containing a substantial part of the amino acid sequence of SEQ. ID. NO. 2” is defined as a polypeptide containing part of the amino acid sequence of SEQ. ID. NO. 2, which is long enough to still have the same function, essential for methionine biosynthesis, as the polypeptide consisting of the amino acid sequence of SEQ. ID. NO. 2. Any polypeptide, as long as it retains the essential function of methionine biosynthesis, satisfies the requirement of the present invention, and thus its length or activity is not important. That is, even if it is lower in activity than the intact polypeptide of SEQ. ID. NO. 2, any polypeptide that has the essential function for methionine biosynthesis may be included within the range of “the polypeptide that contains a substantial part of the amino acid sequence of SEQ. ID. NO. 2”, irrespective of the sequence length thereof. Those who are skilled in the art, that is, those who understand the prior art related to the present invention, expect that a deletion or an addition mutant of a polypeptide containing the amino acid sequence of SEQ. ID. NO. 2 will still retain the methionine synthesis function. As such, a polypeptide that contains the amino acid sequence of SEQ. ID. NO. 2, but from which an N- or C-terminal region has been deleted, is still functional. Generally, it is accepted in the art that even if its N-terminal region or C-terminal region is deleted therefrom, a mutant polypeptide can still retain the function of the intact polypeptide. As a matter of course, if the deleted N- or C-terminal region corresponds to a motif essential for the function of the peptide, the deleted polypeptide loses the function of the intact polypeptide. Nonetheless, the discrimination of such inactive polypeptides from active polypeptides is well known to those skilled in the art. Further, a mutant polypeptide which lacks a portion other than an N- or C-terminal region can still retain the function of the intact polypeptide. Also, those skilled in the art can readily examine whether or not such a deletion mutant still retains the function of the intact polypeptide. Particularly, in light of the fact that the present invention discloses the nucleotide sequence of SEQ. ID. NO. 3 and the amino acid sequence of SEQ. ID. NO. 2 and provides examples in which whether the polypeptide consisting of the amino acid sequence of SEQ. ID. NO. 2, encoded by the nucleotide sequence of SEQ. ID. NO. 3, has a methionine synthesis function was clearly examined, it will be clearly apparent that those who are skilled in the can examine whether a deletion mutant of the polypeptide comprising the amino acid sequence of SEQ. ID. NO. 2 still functions like the intact polypeptide. Accordingly, it must be understood in the present invention that “a polypeptide containing a substantial part of the amino acid sequence of SEQ. ID. NO. 2” means any deletion mutant that can be prepared on the basis of the disclosure of the invention by those skilled in the art and that retains the methionine synthesis function.

As used in the foregoing and the following descriptions, including the claims, the phase “a polypeptide substantially similar to that of (a) or (b)”, means a mutant that has at least one substituted amino acid residue but still retains the function of the amino acid sequence of SEQ. ID. NO. 2, that is, the methionine synthesis function. Likewise, if a mutant in which at least one amino acid residue is substituted still shows the methionine synthesis function, its activity or substitution percentage is not important. Accordingly, no matter how much lower a mutant polypeptide is in activity than a polypeptide containing the intact amino acid sequence of SEQ. ID. NO. 2, or no matter how much a mutant polypeptide has been substituted with amino acid residues compared to a polypeptide containing the intact amino acid sequence of SEQ. ID. NO. 2, the mutant polypeptide is included within the scope of the present invention as long as it shows the methionine synthesis function. Even if it has at least one amino acid residue substituted for a corresponding residue of the intact polypeptide, the mutant polypeptide still retains the function of the intact polypeptide if the substituted amino acid residue is chemically equivalent to the corresponding one. For instance, when alanine, a hydrophobic amino acid, is substituted with a similarly hydrophobic amino acid, e.g., glycine, or with a more hydrophobic amino acid, e.g, valine, leucine or isoleucine, the polypeptide(s) containing such substituted amino acid residue(s) still retain(s) the function of the intact polypeptide, even if it(they) has(have) lower activity. Likewise, a polypeptide(s) containing substituted amino acid residue(s), resulting from substitution between negatively charged amino acids, e.g., glutamate and aspartate, still retains the function of the intact polypeptide, even if it has lower activity. Also, this is true of a mutant polypeptide in which substitution occurs between positively charged amino acids. For example, a substitution mutant polypeptide, containing lysine instead of arginine, still shows the function of the intact polypeptide even if its activity is lower. In addition, polypeptides which contain substituted amino acid(s) in their N- or C-terminal regions still retain the function of the intact polypeptide. It is plainly obvious to those skilled in the art that current technology makes it possible to prepare a mutant polypeptide that retains the methionine synthesis function of the polypeptide containing the amino acid sequence of SEQ. ID. NO. 2, with at least one amino acid residue substituted therein. Also, those skilled in the art can examine whether a substitution mutant polypeptide still retains the function of the intact polypeptide. Further, because the present invention discloses the nucleotide sequence of SEQ. ID. NO. 3 and the amino acid sequence of SEQ. ID. NO. 2 and provides examples in which whether the polypeptide consisting of the amino acid sequence of SEQ. ID. NO. 2, encoded by the nucleotide sequence of SEQ. ID. NO. 3, has a methionine synthesis function was clearly examined, it will be very apparent that “the polypeptide substantially similar to that of (a) or (b)” can be readily prepared by those who are skilled in the art. Accordingly, the “polypeptide substantially similar to that of (a) or (b)” is understood to include all polypeptides that have the methionine synthesis function, in spite of the presence of at least one substituted amino acid therein.

Although “a polypeptide substantially similar to that of (a) or (b)” means any mutant that has at least one substituted amino acid residue but still retains the methionine synthesis function, a polypeptide which shares higher homology with the amino acid sequence of SEQ. ID. NO. 2 is more preferable from the point of view of activity. Useful is a polypeptide that shows 60% or higher homology with the wild-type polypeptide, with the best preference for 100% homology.

In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

Because “the polypeptide substantially similar to that of (a) or (b) includes polypeptides substantially similar to “the polypeptide containing a substantial part of the amino acid sequence of SEQ. ID. NO. 2” as well as polypeptides substantially similar to “the polypeptide having an amino acid sequence 100% coincident with SEQ. ID. NO. 2”, the above description is true both for polypeptides substantially similar to “the polypeptide having the entire amino acid sequence of SEQ. ID. NO. 2” and for polypeptides substantially similar to “the polypeptide containing a substantial part of the amino acid sequence of SEQ. ID. NO. 2”.

In accordance with another aspect thereof the present invention provides an isolated polynucleotide encoding the above-mentioned polypeptide. Herein, the term “the above-mentioned polypeptide” is intended to include not only the polypeptide having the amino acid sequence of SEQ. ID. NO. 2, polypeptides containing a substantial part of the amino acid sequence of SEQ. ID. NO. 2, and polypeptides substantially similar to these peptides, but also all polypeptides that retain the methionine synthesis function in the preferred embodiments. Therefore, the polynucleotide of the present invention includes an isolated polynucleotide encoding a polypeptide that has the methionine synthesis function and contains the entire amino acid sequence of SEQ. ID. NO. 2 or a substantial part of the amino acid sequence thereof, and an isolated polynucleotide encoding a polypeptide substantially similar to such polypeptides. Furthermore, the polynucleotide of the present invention includes all isolated polynucleotides encoding polypeptides that share homology with the amino acid sequence of SEQ. ID. NO. 2. If an amino acid sequence is revealed, a polynucleotide encoding the amino acid sequence can be readily prepared on the basis of the amino acid sequence by those skilled in the art.

In the present invention, the phrase “the isolated polynucleotide” as used herein is intended to include all chemically synthetic polynucleotides, isolated polynucleotides from living bodies, especially Arabidopsis thaliana, and polynucleotides containing modified nucleotides, whether single- or double-stranded RNA or DNA. Accordingly, cDNAs, chemically synthetic polynucleotides, and gDNAs isolated from living bodies, especially Arabidopsis thaliana, fall into the range of “the isolated polynucleotide”. On the basis of the amino acid sequence of SEQ. ID. NO. 2, and the nucleotide sequence of SEQ. ID. NO. 3, encoding the amino acid sequence therefor, and technology known in the art, the preparation of corresponding cDNAs and chemically synthetic polynucleotides and the isolation of gDNA can be readily achieved by those who are skilled in the art.

In accordance with a further aspect thereof, the present invention provides a polynucleotide that contains or is substantially similar to part of the nucleotide sequence of SEQ. ID. NO. 3. Herein, the phrase “a polynucleotide that contains part of the nucleotide sequence of SEQ. ID. NO. 3” means a polynucleotide that has a sequence long enough to identify and/or isolate a gene having the methionine synthesis function in living bodies, especially Arabidopsis thaliana. The phrase “a polynucleotide that is substantially similar to part of the nucleotide sequence of SEQ. ID. NO. 3” means a polynucleotide that contains at least one substituted nucleotide residue, compared to the nucleotide sequence of SEQ. ID. NO. 3, and has sequence-dependent binding ability sufficient to identify and/or isolate a gene having a methionine synthesis function in living bodies, including Arabidopsis thaliana.

As long as the nucleotide sequence of SEQ. ID. NO. 3 is disclosed, the identification and/or isolation of a gene having the methionine synthesis function in Arabidopsis thaliana or other organisms can be readily carried out on the basis thereof by those skilled in the art.

Accordingly, the polynucleotide of the present invention is intended to include all polynucleotides which have a sequence length or sequence-dependent binding power sufficient to identify and/or isolate a gene having the methionine synthesis function in living bodies including Arabidopsis thaliana, irrespective of the length and sequence homology to the nucleotide sequence of SEQ. ID. NO. 3.

In order to be used as a probe for examining whether or not an unknown gene has the same nucleotide sequence as that of a known gene or for isolating an unknown gene, a polynucleotide is generally known to have to contain 30 or more consequent nucleotide residues. Thus, the polynucleotide of the present invention preferably includes 30 or more consequent nucleotide residues out of the nucleotide sequence of SEQ. ID. NO. 3. Nevertheless, a poly (or oligo) peptide consisting of 30 or fewer consequent nucleotide residues out of the nucleotide sequence of SEQ. ID. NO. 3 is still included within the scope of the present invention. The reason is that the poly (or oligo) nucleotide, although short, is sufficient to identify and/or isolate a gene having the methionine synthesis function from Arabidopsis thaliana or other organisms if it shares 100% homology with part of the nucleotide sequence of SEQ. ID. NO. 3 and the identification and/or isolation conditions (buffer pH, concentration, etc.) are stringent. Based on the disclosure of the present invention, herein, those skilled in the art can readily construct and detect a polynucleotide which is 30 or fewer bases long in order to identify and/isolate a gene having the methionine synthesis function from Arabidopsis thaliana or other organisms, and can readily identify and/or isolate a gene having the methionine synthesis function from Arabidopsis thaliana or other organisms using the constructed polynucleotide.

In accordance with still a further aspect thereof, the present invention provides an antisense nucleotide able to complementarily bind to the above-mentioned polynucleotide.

The antisense nucleotide is intended to include all poly (or oligo) nucleotides that complementarily bind to the above-mentioned polynucleotide to inhibit transcription (when the polynucleotide is DNA) or translation (when the polynucleotide is RNA). If the antisense nucleotide can complementarily bind to the polynucleotide encoding the polypeptide having the methionine synthesis function to inhibit the transcription or translation of the polynucleotide (DNA or RNA, respectively), its length or homology to a complementary sequence is not important. A polynucleotide, even if short, e.g., 30 nucleotides long, can function as an antisense nucleotide as long as it shares 100% homology with a sequence complementary to the gene of interest (DNA or RNA) and stringent conditions including buffer concentration and pH are observed. Additionally, although it does not share 100% homology with a complementary sequence of the gene of interest, a polynucleotide may be used as an antisense nucleotide if it has a suitable length. Therefore, it should be noted that as long as it can inhibit the transcription or translation of a gene of interest, any poly (or oligo) nucleotide is included in the range of the antisense nucleotide of the present invention, irrespective of length and homology to a complementary sequence. On the basis of the nucleotide sequence of SEQ. ID. NO. 3 and the amino acid sequence of SEQ. ID. NO. 2, those skilled in the art can readily determine the length and homology necessary for an antisense nucleotide, and can prepare such an antisense nucleotide using current technology.

Preferable is the antisense nucleotide, the complete or partial sequence of which is complementary to a length of the nucleotide sequence of SEQ. ID. NO. 3. In light of the previous description, herein, the phrase “complementary to a length of the nucleotide sequence of SEQ. ID. NO. 3” should be understood to mean a sequence long enough to bind to DNA comprising the nucleotide sequence of SEQ. ID. NO. 3 or to an RNA transcripted from the DNA so as to inhibit the transcription or translation of the polynucleotide.

In accordance with still another aspect thereof, the present invention provides a method for inhibiting the growth of plants. The method comprises suppressing the expression or activity of the polypeptide, based on the amino acid sequence of SEQ. ID. NO. 2 or a similar amino acid sequence, having the methionine synthesis function. As described above, methionine is a vitamin essential for the growth of both plants and animals, and its biosynthesis pathway in which a non-vitamin B12-dependent methionine synthesis enzyme plays a pivotal role exists in plants, but not in animals. Thus, the suppression of the expression or activity of the polypeptide having the methionine synthesis function leads to the suppression of the growth of plants, without injuring animals.

The polypeptide of the present invention functions as a vitamin B12-independent methionine synthesis enzyme which is essential for methionine biosynthesis. Therefore, if the expression of the polypeptide of the invention is suppressed, methionine synthesis is blocked, resulting in the inhibition of plant growth without injure to humans or animals. In practice, when an antisense nucleotide sequence complementary to the nucleotide sequence of SEQ. ID. NO. 3 is introduced into Arabidopsis thaliana to inhibit the activity of the polypeptide of the present invention, as will be elucidated later, the transformed Arabidopsis thaliana is found to be significantly inhibited from growing, even to the death. Thus, the method for inhibiting the growth of plants in accordance with the present invention can be accomplished by suppressing the expression or activity of the polypeptide of the present invention.

By the term “inhibition of plant growth, as used in the foregoing and the following descriptions the claims, it is meant that a plant is killed or decreases in biomass compared to the wild-type.

As used in the foregoing and the following descriptions the claims, the phrase “a polypeptide consisting of an amino acid sequence similar to that of SEQ. ID. NO. 2” is intended to include all polypeptides that are homologs of the polypeptide of SEQ. ID. NO. 2, with the retention of the vitamin B12-independent methionine synthesis function, and are different in amino acid sequence from the polypeptide of SEQ. ID. NO. 2 due to evolutionary differences between plants. In the method for inhibiting the growth of plants in accordance with the present invention, the plants include all types of plants as well as Arabidopsis thaliana although the polypeptide consisting of the amino acid sequence of SEQ. ID. NO. 2 was isolated from Arabidopsis thaliana. More preferable from the point of view of activity is a polypeptide consisting of an amino acid sequence similar to that of SEQ. ID. NO. 2, which shares higher homology with the amino acid sequence of SEQ. ID. NO. 2. Useful is a polypeptide that shows 60% or higher homology with the wild-type polypeptide, with the best preference for 100% homology. In more detail, more preferable are sequence homologies of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% and 99%, in ascending order of preference.

By the phrase “suppression of the polypeptide of SEQ ID NO. 2 or a base sequence similar thereto”, as used in the foregoing and the following descriptions including the claims, it is meant that the production of the polypeptide is inhibited by suppressing the expression of the gene encoding the polypeptide or the polypeptide is inactivated using a chemical.

The suppression of polypeptide expression can be achieved using various methods that are well known in the art, including antisense nucleotide introduction, gene deletion, gene insertion, T-DNA introduction, homologous recombination, transposon tagging, and RNA silencing with siRNA (small interfering RNA).

In the following examples, antisense nucleotide introduction was utilized to suppress the growth of plants. In detail, an antisense nucleotide to a polynucleotide consisting of the nucleotide sequence of SEQ. ID. NO. 3 was prepared and inserted into a vector. The recombinant vector (pSEN-antiAtMSG) thus constructed was introduced into Agrobacterium tumefaciens, which was then transfected into Arabidopsis thaliana. Seeds from the resulting mutant Arabidopsis thaliana were found to grow in a significantly delayed manner (see Example 2).

In the method for suppressing the growth of plants, an antisense nucleotide complimentary to part of the nucleotide sequence of SEQ. ID. NO. 3 is preferably introduced into plants. More preferably, a transformant harboring a recombinant vector carrying the antisense nucleotide is introduced into plants. Most preferably, the transformant is the Agrobacterium tumefaciens transformed with the recombinant vector. Herein, the phrase “complementary to part of the nucleotide sequence of SEQ. ID. NO. 3” has the same meaning as in the description of the antisense nucleotide.

Generally, an antisense nucleotide is known to bind to a target nucleotide in nucleic acids (RNA or DNA) to suppress the function or synthesis of the nucleic acids. With the ability to hybridize both RNA and DNA, an antisense nucleotide corresponding to a target gene can inhibit the expression of the target gene in the transcription or translation level thereof.

Accordingly, the suppression of the expression or activity of a polypeptide consisting of the amino acid sequence of SEQ. ID. NO. 2 or a similar amino acid sequence results in the suppression of the growth of plants.

In accordance with yet still another aspect of the present invention, a method for screening a material suppressive of the growth of plants is provided. This method comprises detecting a material that suppresses the expression or activity of the polypeptide consisting of the amino acid sequence of SEQ. ID. NO. 2 or a similar amino acid sequence and having the methionine synthesis function.

Herein, the phrase “the polypeptide consisting of the amino acid sequence of SEQ. ID. NO. 2 or a similar amino acid sequence” has the same meaning as in the description of the method for suppressing the growth of plants.

For the same reason as in the description of the method for suppressing the growth of plants, the material suppressive of the expression of the polypeptide is preferably an antisense nucleotide complementary to part of the nucleotide sequence of SEQ. ID. NO. 3, more preferably a transformant harboring a recombinant vector carrying the antisense nucleotide, and still more preferably Agrobacterium tumefaciens transformed with the recombinant vector. Herein, the phrase “complementary to a part of the nucleotide sequence of SEQ. ID. NO. 3” has the same meaning as in the description of the antisense nucleotide.

In accordance with yet still an additional aspect of the present invention, a material suppressive of the growth of plants, obtained through the screening method, is provided.

As such, an antisense nucleotide complementary to part of the nucleotide sequence of SEQ. ID. NO. 3, a recombinant vector carrying the antisense nucleotide, and Agrobacterium tumefaciens transformed with the recombinant vector may be exemplary.

### Advantageous Effects

As described above, the present invention provides a polypeptide having a methionine synthesis function, a polynucleotide encoding the polypeptide, an antisense nucleotide complementary to the polynucleotide, a recombinant vector carrying the polynucleotide, a transformant harboring the recombinant vector, a method for suppressing the growth of plants, a method for screening material that suppresses the growth of plants, and material that suppresses the growth of plants.

### DESCRIPTION OF DRAWINGS

FIG. 1 is a schematic diagram of a pSEN vector into which a polynucleotide encoding a polypeptide having a methionine synthesis function, particularly a polynucleotide of SEQ ID No. 1, will be inserted in an antisense direction

FIG. 1 is a schematic diagram showing the structure of the recombinant vector pSEN-antiAtMSG, prepared by inserting a polynucleotide encoding a polypeptide having a methionine synthesis function, particularly a polynucleotide of SEQ. ID. NO. 1, into the vector pSEN in an antisense direction.

FIG. 3 is a photograph showing mutant Arabidopsis thaliana grown from T1 seeds of Arabidopsis thaliana transformed with the recombinant vector pSEN-antiAtMSG of FIG. 2. In FIG. 3, AtMSG shows the transformed T1 Arabidopsis thaliana and Col-O is a wild-type Arabidopsis thaliana.

FIG. 4 is a photograph showing wild-type Arabidopsis thaliana grown for 18 days and 32 days after germination and mutant Arabidopsis thaliana grown for 18 days and 32 days after germination from T2 seeds of Arabidopsis thaliana transformed with the recombinant vector pSEN-antiAtMSG (bar indicates 1 cm). In FIG. 4, 18 d-old AtMSG and 32 d-old AtMSG stand for mutant Arabidopsis thaliana grown for 18 days and 32 days from T2 seeds, respectively, and 18 d-old Col-O and 32 d-old Col-O stand for wild-type Arabidopsis thaliana grown for 18 days and 32 days, respectively.

FIG. 5 is a photograph showing wild-type Arabidopsis thaliana grown for 18 days and 32 days after germination, mutant Arabidopsis thaliana grown for 18 days after germination from T2 seeds of Arabidopsis thaliana transformed with the recombinant vector pSEN-antiAtMSG, and mutant Arabidopsis thaliana grown for 32 days in total after germination, resulting from the treatment of the 18-day-old transformed Arabidopsis thaliana with methionine for 14 days (bar indicates 1 cm). In FIG. 5, 18-old AtMSG and 32 d-old AtMSG stand for mutant Arabidopsis thaliana grown for 18 days from T2 seeds and 32 days in total after germination from T2 seeds, resulting from the treatment of the 18-day-old transformed Arabidopsis thaliana with methionine for 14 days, respectively and 18 d-old Col-O and 32 d-old Col-O showed wild-type Arabidopsis thaliana grown for 18 days and 32 days, respectively.

### BEST MODE

A better understanding of the present invention may be obtained through the following examples, which are set forth to illustrate but are not to be construed as the limit of the present invention.

### Example 1

### Isolation of a Gene Encoding a Polypeptide Having a Methionine Synthesis Function from Arabidopsis thaliana

A screening process was performed for isolating a gene, encoding a polypeptide having a methionine synthesis function, from Arabidopsis thaliana.

### Example 1-1

### Cultivation and Nurturance of Arabidopsis thaliana

Arabidopsis thaliana was cultured in soil in pots or in an MS medium (Murashige and Skoog salts, Sigma, USA) containing 2% sucrose (pH 5.7) and 0.8% agar in Petri dishes. When using pots, the plants were cultivated at 22° C. under a light-dark cycle of 16/8 hours in a growth chamber.

### Example 1-2

### RNA Isolation and cDNA Library Construction

In order to construct Arabidopsis thaliana cDNA libraries, first, total RNA was isolated from Arabidopsis thaliana leaves in various stages of differentiation using a TRI reagent (Sigma, U.S.A.). poly(A)+ RNA was purified from the isolated total RNA using an mRNA purification kit (Pharmacia, U.S.A.) according to the enclosed instructions for the protocol. Double-stranded cDNA was prepared from the poly(A)+ RNA with the aid of a cDNA synthesis kit (Time Saver cDNA synthesis kit, Pharmacia, U.S.A.), with NotI-(dT)₁₈ serving as a primer.

### Example 1-3

### Isolation of a Gene Encoding a Polypeptide Having a Methionine Synthesis Function

Based on the amino acid sequence of a putative vitamin B12-independent methionine synthesis enzyme (GenBank accession number NM 180176) of Arabidopsis thaliana, a sense primer, represented by SEQ. ID. NO. 4, containing an BstEII site, and an antisense primer, represented by SEQ. ID. NO. 4, containing a BglII site, were synthesized. Using these two primers, a full length cDNA containing a 5′- and a 3′-UTR was amplified through PCR (polymerase chain reaction) from the cDNA library constructed in Example 1-2.

The cDNA was analyzed to have a 2,298 bp open reading frame (ORF) of SEQ ID NO. 3, comprised of ten exons, encoding a polypeptide consisting of 765 amino acid residues with a molecular weight of about 84.6 kDa, and was called AtMSG (Arabidopsis thaliana methionine synthase in Genomine) or AtMSG gene. Its protein is expressed as “AtMSG” or “AtMSG protein. The AtMSG protein encoded by the gene was found to have an isoelectric point of 6.47.

### Example 2

### Preparation and Characterization of Arabidopsis thaliana Mutant Harboring Antisense Construct Complementary to AtMSG Gene

### Example 2-1

### Preparation of Arabidopsis thaliana Mutant Harboring Antisense Construct Complementary to AtMSG Gene

In order to examine whether the gene is involved in methionine synthesis and causes a mutation in the phenotype of plants, the AtMSG gene was introduced in the antisense direction into Arabidopsis thaliana to suppress the expression of the AtMSG transcript.

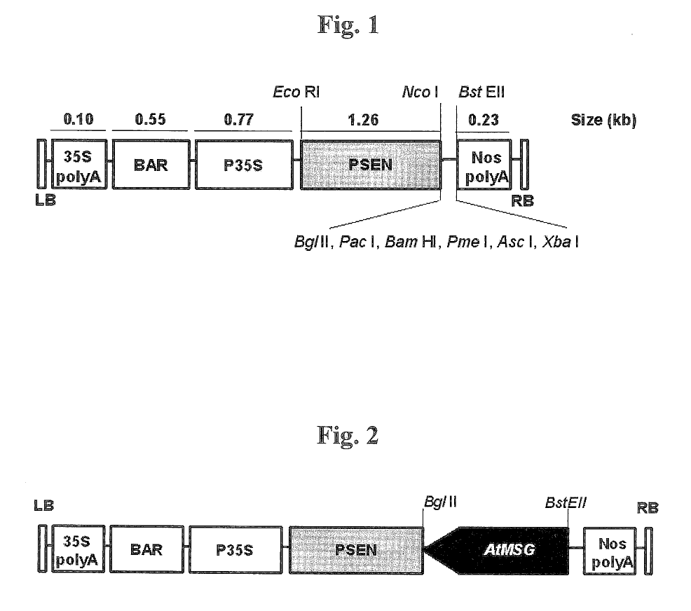

AtMSG cDNA containing 5′- and 3′-UTR was amplified from the cDNA library of Arabidopsis thaliana through PCR using a sense primer, represented by SEQ. ID. NO. 4, containing an BstEII site, and an antisense primer, represented by SEQ. ID. NO. 5, containing a BglII site. The PCR product thus obtained was digested with restriction enzymes BglII and BstEII and inserted in an antisense direction into the pSEN vector, under the control of a sen1 promoter, a stress or senescence-associated gene, to construct a recombinant vector, named pSEN-antiAtMSG, harboring an antisense construct complementary to the AtMSG gene. The sen1 promoter shows specificity for the genes expressed according to growth stage. FIGS. 1 and 2 respectively show the structures of the pSEN vector and the pSEN-antiAtMSG recombinant vector prepared by introducing the AtMSG gene in an antisense direction into the pSEN vector. In FIGS. 1 and 2, BAR stands for a bar gene (phosphinothricin acetyltransferase gene) conferring Basta resistance, RB for a right border, LB for a left border, P35S for a CaMV 35S RNA promoter, 35S poly A for CaMV 35S RNA poly A, PSEN for a sen1 promoter, and Nos polyA for nopaline synthase gene polyA.

The pSEN-antiAtMSG recombinant vector was introduced into Agrobacterium tumefaciens using an electroporation method. The transformed Agrobacterium strain was cultured at 28° C. to an O.D.₆₀₀ of 1.0, followed by harvesting cells by centrifugation at 25° C. at 5,000 rpm for 10 min. The cell pellet thus obtained was suspended in an infiltration medium (IM: 1× MS SALTS, 1× B5 vitamin, 5% sucrose, 0.005% Silwet L-77, Lehle Seed, USA) until O.D.₆₀₀ reached 2.0. Four week-old Arabidopsis thaliana was immersed in the Agrobacterium suspension in a vacuum chamber and allowed to stand for 10 min under a pressure of 10⁴ Pa. Thereafter, the Arabidopsis thaliana was placed for 24 hours in a polyethylene bag. The transformed Arabidopsis thaliana was grown to obtain seeds (T1). Wild-type Arabidopsis thaliana was used as a control.

### Example 2-2

### Characterization of Transformed T1 and T2 Arabidopsis thaliana

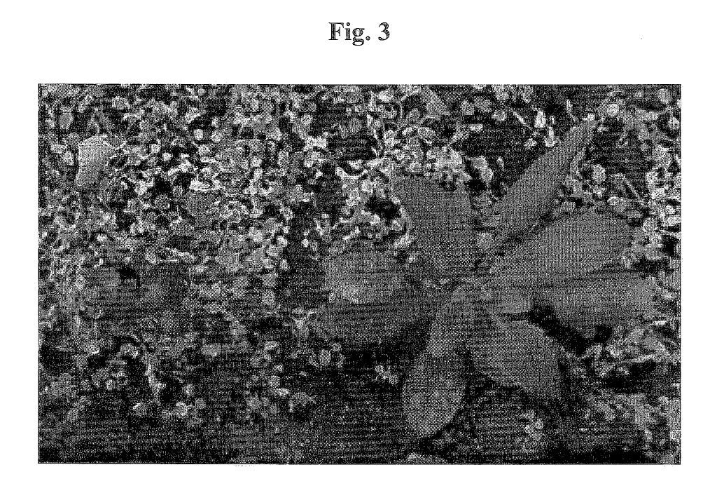

After being immersed in a 0.1% Basta herbicide solution (Kyung Nong Co. Ltd., Korea) for 30 min, seeds from the Arabidopsis thaliana transformed in Example 2-1 were cultured. A Basta herbicide was applied five times to each pot in which the transformed Arabidopsis thaliana grew, and observation was made of the growth pattern of the Arabidopsis thaliana in each pot. Compared to the control (wild-type Arabidopsis thaliana), the Arabidopsis thaliana transformed with the pSEN-antiAtMSG recombinant vector showed various phenotype mutations including retarded growth, which were believed to result from difference in the suppressive activity of the antisense gene against gene expression from one individual to another. Representative examples of the phenotype mutations were as follows. First, the plants were significantly suppressed from growing. Another phenotype mutation was found in leaf morphology and color. The leaves of the transformed Arabidopsis thaliana grew circular whereas the control has oval leaves. Further, the transformed Arabidopsis thaliana had overlapped leaves due to undifferentiated petioles. The leaves turned dark brown leaves and suffered from etiolation due to the retarded formation of chlorophyll and the accumulation of anthocyanin. Moreover, the insufficient differentiation of flow stalks led to significantly short statures over the transformed plants and caused significant hindrance in seed formation. Finally, the potent antisense effect on the gene of the present invention caused death of the plant transformant, as well as morphological aberration and growth suppression (FIG. 3).

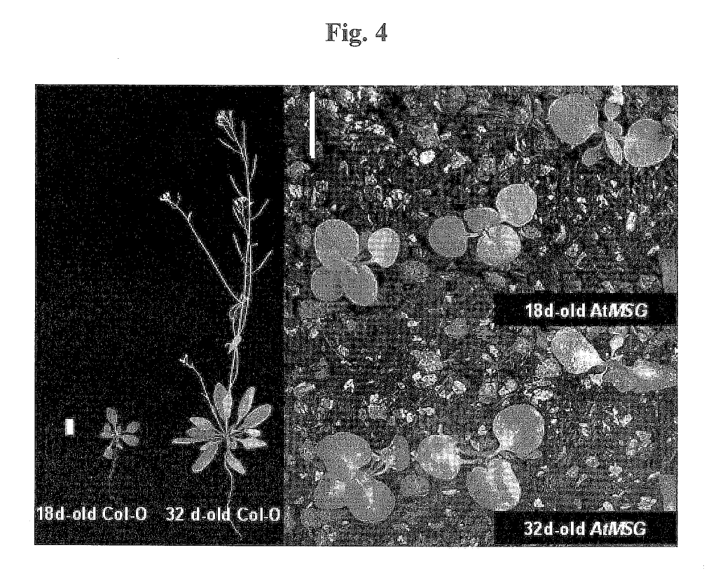

The phenotype of Arabidopsis thaliana transformed with an antisense construct of the AtMSG gene was examined. T2 seeds were obtained from the T1 line of the transformed Arabidopsis thaliana. For this, T2 seeds, which had been subjected to low temperature treatment (4° C.) for 3 days, were cultured in pots. Phenotype mutations of the individual plants cultured for 18 days after germination were as follows. Leaves were greatly suppressed from coming out after seed leaf production. Leaf differentiation was observed to further proceed no more after two leaves in some line and after four leaves in other lines. As for leaf morphology, leaves seemed to overlap due to insufficient petiole differentiation and suffered from the morphological aberration of growing circular rather than oval. Next, the transformed plants were significantly suppressed from growing so that they grew to a size less than 1/10 that of the control (FIG. 4). In order to examine the persistence of the phenotypic change, the transformed Arabidopsis thaliana were observed for phenotype properties for 32 days after germination. Most individuals did not grow further after 18 days and generally showed a mortal phenotype. Particularly, the mortal phenotype of etilolation and emanciation resulting from the suppression of chlorophyll production and the accumulation of anthocyanin was common to the transformed plants (FIG. 4). The phenotype mutations of the transformed individuals were believed to be attributed to the gene suppression against methionine biosynthesis according to the present invention. Therefore, the gene of the present invention is inferred to be a gene essential for the growth and development of plants.

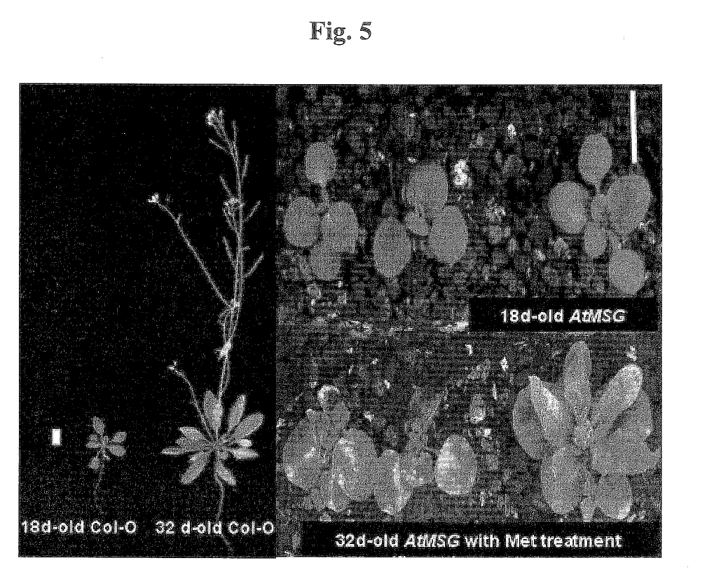

As described above, the AtMSG gene was inferred to encode a protein having an enzymatic function essentially involved in the methionine biosynthesis pathway. To investigate this, the transformed Arabidopsis thaliana was cultured for 32 days in total: the plants 18 days old after germination (FIG. 5) were cultured for 14 days in a medium containing methionine (Sigma USA) at a concentration of 1 mg/100 mL. Compared to the Arabidopsis thaliana treated with no methionine, the Arabidopsis thaliana treated with methionine were found to recover the wild-type phenotype from a mortal phenotype. When treated with methionine, the mortal phenotype (FIG. 5) differentiated new leaves in a bushy form from growing points. In the newly grown leaves, etiolation and withering were not observed (FIG. 5), which implies that the treatment of the transformed Arabidopsis thaliana with methionine leads to phenotype recovery. Therefore, the plants transformed with an antisense construct of the AtMSG gene were identified to be a methionine auxotroph, suggesting that the polynucleotide encoded by the gene of the present invention might be an efficient target for the development of plant growth regulators or herbicides.

### SEQUENCE LIST PRETEXT

Sequence list Attached

1. A polypeptide, serving as a vitamin B12-independent methionine synthesis enzyme having an essential function for methionine biosynthesis, selected from the group consisting of:
(a) a polypeptide having an entire amino acid sequence of SEQ. ID. NO. 2;
(b) a polypeptide containing a substantial part of the amino acid sequence of SEQ. ID. NO. 2; and
(c) a polypeptide substantially similar to that of (a) or (b).

2. An isolated polynucleotide, encoding the polypeptide of claim 1.

3. An antisense nucleotide sequence, complementary to the polynucleotide of claim 2.

4. A recombinant vector carrying the antisense nucleotide sequence of claim 3.

5. An Agrobacterium tumefaciens mutant, transformed with a recombinant vector harboring the antisense nucleotide sequence of claim 3.

6. A method for inhibiting growth of plants, comprising a step of suppressing the expression or activity of a polypeptide having a methionine synthesis function, the polypeptide having an amino acid sequence 100% coincident with or similar to SEQ. ID. NO. 2.

7. The method as defined in claim 6, wherein the suppressing step comprises the introduction of the antisense nucleotide sequence of claim 3 into the plants.

8. The method as defined in claim 6, wherein the suppressing step is carried out using a technique selected from the group consisting of gene deletion, gene insertion, T-DNA introduction, homologous recombination, transposon tagging, RNA silencing using siRNA, and combinations thereof.

9. A method for screening a material suppressive of growth of plants, comprising the step of detecting a material suppressive of expression or activity of a polypeptide having an amino acid sequence 100% coincident with or similar to SEQ. ID. NO. 2.

10. A material suppressive of growth of plants, obtained using the method of claim 9.

11. The material as defined in claim 10, wherein the material is selected from a group consisting of the antisense nucleotide of claim 3, a recombinant harboring the antisense nucleotide vector of claim 3, and an Agrobacterium tumefaciens mutant transformed with a recombinant vector harboring the antisense nucleotide of claim 3.
